(19) United States
(12) Patent Application Publication
(10) Pub. No.: US 2007/0098757 A1
(43) Pub. Date: May 3, 2007

(54) E-PTFE foil impregnated with an encapsulated bioactive substance

(76) Inventors: Doris Klee, Aachen (DE); Christoph Hilgers, Eschweiler (DE)

Correspondence Address: MORRISON & FOERSTER, LLP, 555 WEST FIFTH STREET, SUITE 3500, LOS ANGELES, CA, 90013-1024, US

(21) Appl. No.: 11/637,431

(22) Filed: Dec. 12, 2006

Related U.S. Application Data
(62) Division of application No. 10/381,381, filed on Aug. 12, 2003.

(30) Foreign Application Priority Data
Sep. 28, 2000 (GB) GB00238071

(51) Int. Cl. A61F 2/02

(52) U.S. Cl. 424/423; 977/900

(57) ABSTRACT

A method of impregnating an e-PTFE (expanded polytetrafluoroethylene) foil with a biologically-active substance.

### PRIORITY

This application is a division of U.S. patent application Ser. No. 10/381,381, filed Aug. 12, 2003, which is a national stage application under 35 USC § 371 of International Patent Application No. PCT/EP2001/11267, filed Sep. 28, 2001, claiming priority to United Kingdom Patent Application No. 00238071.1, filed Sep. 28, 2000, each of which is incorporated by reference into this application as if fully set forth herein.

### BACKGROUND

This invention relates to an e-PTFE (expanded polytetrafluoroethylene) foil impregnated with a biologically-active substance, to a method of so impregnating a foil and to a prosthesis comprising an e-PTFE impregnated foil, thereby carrying a releasable biologically-active substance.

Prostheses, such as stent grafts, can usefully include an e-PTFE membrane, this being inert in the body and capable of providing a useful barrier function. In recent years it has been a challenge to manufacturers of prostheses to invent ways of incorporating biologically active substances in stents, grafts and other prostheses. Growth factors are one such biologically-active substance which would be advantageous to incorporate. Others may include cellular proliferation-controlling or migration-controlling agents, and agents to inhibit thrombosis. The innumerable interstices in an e-PTFE foal (otherwise called herein “membrane” or “film”) provide an attractive location for the placement of biologically active substances, but a method has to be found how to load the interstices with the active substance. The present invention aims to provide one route to achieve such loading.

U.S. Pat. No. 5,480,711 discloses nano-porous PTFE and its use as a biomaterial. U.S. Pat. No. 5,716,660 discloses e-PTFE prostheses impregnated with a solid insoluble biocompatible material, specifically an extracellular matrix protein, such as collagen or gelatin. U.S. Pat. No. 5,972,027 proposes to load a porous stent with a biomaterial, such as a drug. The drug may be carried in solution and loaded into the pores by imposing a pressure gradient on the solution. The stent is to be made from a metal powder, but the possibility of a stent made from PTFE powder is also mentioned. EP-A-0706376 discloses use of taxol in the manufacture of a stent. Taxol is disclosed as an anti-angiogenic factor.

### BRIEF SUMMARY OF THE INVENTION

It is an object of the present invention to provide e-PTFE foil impregnated with a biologically-active substance. It is another object of the present invention to provide a method of impregnating an e-PTFE foil with a biologically-active substance. It is yet another object of the present invention to provide a prosthesis, such as a stent, comprising an impregnated e-PTFE foil carrying a releasable biologically-active substance.

In accordance with one aspect of the present invention, there is provided a method of impregnating an e-PTFE foil with a biologically-active substance which involves imposing across the foil a pressure differential sufficient to urge into the interstices of the foil a suspension of nanoparticles in a fluid medium, the nanoparticles containing a desired biologically-active substance. In another aspect, the present invention provides an e-PTFE foil so impregnated. In yet another aspect, the present invention provides a prosthesis comprising an e-PTFE foil so impregnated.

Taking into account the typical dimensions of interstices in e-PTFE foils, the range of sizes of nanoparticles which lend themselves to such impregnation will generally lie in a range of from 10 nm to 5 μm for average diameters of typically spherical nanoparticles (nanospheres). A preferred range of diameters is from 100 to 800 nm.

Conveniently, the nanoparticles have a surface layer which encapsulates the active substance of interest, and the surface layer is conveniently bioabsorbable and can be of a lactide-containing polymer, such as poly(D,L-lactic-acid), poly(D,L-lactic-acid-co-glycolide) and poly(D,L-lactic-acid-co-trimethylenecarbonate), the latter hereinafter abbreviated to poly(D,L-lactide-co-TMC).

Once the nanoparticles have been urged into the foil interstices, the biologically-active substance delivered by the nanoparticles should be anchored there. One way which the present inventors have found to anchor the nanoparticle load is to perform a heat treatment of the impregnated foil to agglomerate the nanoparticles. Another is to perform a CO₂ procedure in accordance with the CESP process described in Advanced Engineering Materials 1999, Vol. 1, No. 3-4, pages 206 to 208 in the paper entitled, “Microporous, resorbable implants produced by the CESP process” by Walter Michaeli and Oliver Pfannschmidt of the “Institut fur Kunststoffverarbeitung, Rheinisch-Westflische Technische Hochschule, D-52062 Aachen, Germany,” the content of which paper being incorporated in this specification by this reference. A copy is annexed to the priority document, that is, GB 0023807.1.

For a discussion of preparation techniques and mechanisms of formation of biodegradable nanoparticles from preformed polymers, the reader may refer to a paper by Quintanar-Guerrero, Alleman, Fessi and Doelker which appears in Drug Development and Industrial Pharmacy, 24(12), 1113-1128 (1998). The content of this paper is also incorporated in this specification by this reference. A copy is annexed to the priority document, that is, GB 0023807.1.

In a nutshell, the method preferred herein involves a nanoparticle production step followed by a loading step to impregnate an e-PTFE foil in a pressure cell with a suspension of nanoparticles, followed by an anchoring step to fix in the foil the biologically-active substance carried into the foil by the nanoparticles.

Conveniently, polymeric nanoparticles are manufactured by a solvent evaporation or solvent displacement technique. Loading of the nanoparticles with a medicament or other biologically-active substance is carried out during the nanoparticle manufacturing step.

With more specificity, a base polymer which can be a poly(D,L-lactide), poly(D,L-lactide-co-glycolide) or a poly(D,L-lactide-co-trimethylenecarbonate) is dissolved in a solvent. The solvent can be a water-miscible solvent, such as acetone, when the method is a solvent displacement method. Alternatively, the solvent can be a water-non-miscible solvent, such as CH₂Cl₂, when the technique is a solvent evaporation technique.

In a second step, the biologically-active substance, such as a drug or medicament, is dissolved in or disbursed in the polymer solution and then the solution is poured into an aqueous phase with continuous stirring. The aqueous phase will likely contain a surfactant or a stabiliser. Afterwards, the solvent is vacuum-evaporated from the aqueous phase.

The resulting suspension of nanoparticles is transferred into a pressure chamber with continuous stirring. Continuing the stirring, a pressure differential conveniently in the range up to 10 bar is imposed within the pressure cell across a foil workpiece in order that the pressure differential shall drive the suspension into the interstices of the e-PTFE which forms the foil. The procedure is repeated, as many times as is necessary, in order to load the foil workpiece with the specified quantity of nanoparticle material.

For anchoring the nanoparticle material within the interstices of the foil, the impregnated foil can be treated at an elevated temperature to bring about agglomeration or melting of the nanoparticles within the interstices. Alternatively, a treatment with supercritical CO₂ can be utilized. The supercritical CO₂ dissolves the nanoparticles, thereby causing it to flow so that the nanoparticles lose their discrete shape to form a molten structure within the e-PTFE foil. One way of obtaining this molten structure is to follow the procedures advocated by RWTH-Aachen in its CESP process, mentioned above.

It will be appreciated that the loading of the e-PTFE foil with nanoparticles can be accomplished with foil material to be later incorporated into a medical device, or can be incorporated into foil which has already been incorporated in a medical device. The medical device can be a prosthesis, such as a vascular prosthesis. A vascular prosthesis of special interest for the inventors is a vascular stent. The invention will likely find applications for stents which are not vascular stents, such as biliary, ureteral, uretheral, oesophageal, tracheo-bronchial, colorectal, prostatic, hepatic stents.

The pressure differential imposed on the foil in the pressure cell can be achieved by positive pressurization of the nanoparticle suspension on the upstream side of the foil, or by imposing a sufficiently low enough vacuum on the downstream side of the foil workpiece. The fluid medium within which the nanoparticles are suspended for the loading step in the pressure cell need not be the same fluid medium in which the nanoparticles are created.

The encapsulation of the biologically-active substance, such as a drug or medication, within the nanoparticles need not be with biodegradable synthetic polymeric materials, such as poly-lactide and its co-polymers, polyesther, polyether, polycyanoacrylate, polyhydroxycarboxylic acid, polyanhydride, polyaminoacids, polyhydroxyalkanoate, but could instead be with bio-compatible, non-biodegradable materials through which, for example, the drug diffuses into the body of the patient. To be considered for this task are, for example, biologically-compatible synthetic polymeric substances. These include silicone, polyalcane, polytetrafluorethylene (PTFE, e-PTFE), polyethylene, such as ultra-pure polyethylene (HOSTALON™ (GUR), LUPULEN™ (UHM)), polypropylene, polyester (such as polyethylene terephthalate or DACRON™, TERYLENE™), polyurethane, as well as polyamides, such as NYLON™, aliphatic and aromatic polyamides (NOMEX, KEVLAR).

### BRIEF DESCRIPTION OF THE FIGURES

The present invention will be described, by way of example, with reference to the accompanying figures, of which:

FIGS. 1A and 1B show REM pictures of an untreated and unloaded e-PTFE sample at two magnifications (1A: 2500 fold; 1B: 5000 fold);

FIGS. 2A and 2B show REM pictures of an e-PTFE layer loaded with nanospheres at two magnifications (2A: 2500 fold; 2B: 5000 fold);

FIGS. 3A and 3B show REM pictures of an e-PTFE layer after CO₂ treatment (3A: 2500 fold; 3B: 5000 fold);

FIGS. 4A and 4B show REM pictures of an e-PTFE layer loaded with nanospheres after CO₂ treatment (4A: 2500 fold; 4B: 5000 fold);

FIG. 5 shows a diagram depicting a release pattern of dexamethasone-loaded nanospheres out of poly-(D,L-lactide-co-TMC) (90:10);

FIG. 6 shows UV-spectra of dexamethasone-containing PVA-solution;

FIG. 7 shows a calibration line for dexamethasone in a 4% PVA-solution (M=61000);

FIG. 8 shows UV-spectra of wash solution contaminated with dexamethasone;

FIG. 9 shows a calibration line for dexamethasone in water.

### DETAILED DESCRIPTION OF THE PREFERRED EMBODIMENTS

The production of biodegradable spherical nanoparticles by an (o/w)-emulsion-solvent evaporation technique is described below.

### Particle Production

Spherical nanoparticles made of poly(D,L-lactic-acid-co-trimethylen-ecarbonate) (90:10) were prepared by an (o/w)-emulsion-solvent evaporation technique. 200 mg of the polymer, 40 mg of one model substance dexamethasone in 5 ml of methylenechloride were mixed and cooled down to 0° C. 50 ml of an aqueous 4% (w/v) polyvinylalcohol solution (M=61.000) were added. This mixture was dispersed for 30 s by vortex (Ika Ultra Turrax 25 T basic, Staufen im Breisgau, Germany) at 150.000 rpm at 0° C. and then sonicated using an ultrasound generator (Branson Sonifier 450, Danbury, Conn., USA) for 120 s to produce the oil in water emulsion. The solution was poured into an 500 ml flask and the organic phase was removed for 30 min under stepwise reduction of the pressure (room pressure down to >60 mbar).

### Particle Loading

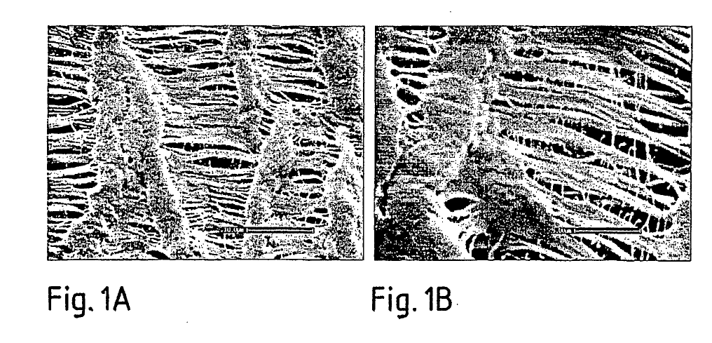

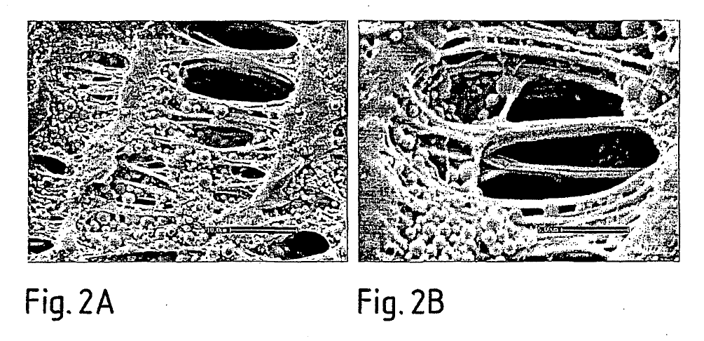

Next, the nanosphere suspension was directly used to incorporate the spheres into the e-PTFE layer by pressing the nanosphere suspension under pressure through an e-PTFE covered stent to obtain a loading of the foil with nanoparticles. The stent was freeze-dried and analysed with REM. FIGS. 1A and 1B show REM pictures of the untreated and unloaded e-PTFE foil in 2500 fold (FIG. 1A) and 5000 fold magnification (FIG. 1B). FIGS. 2A and 2B show the e-PTFE layer loaded with nanospheres at magnification levels of 2500 fold (FIG. 2A) and 5000 fold (FIG. 2B), respectively.

The particle size distribution of the remaining suspension and the filtrate were measured (refer to Table 1 below) using a particle sizer (Malvern instruments, Mastersizer 2000 with dispersing unit Hydro 2000, Worcestershire, Great Britain). Table 1 shows the computed diameter d of the median particle (n=0.5) of the particle distribution.

TABLE 1
d (n = 0.5) values of the remaining suspension and
filtrate compared with the initial d (n = 0.5) value.

| Initial Suspension | Remaining Suspension | Filtrate |
|---|---|---|
| d (n = 0.5) in nm | d (n = 0.5) in nm | d (n = 0.5) in nm |
| 542 | 991 | 256 |

The remaining suspension shows a higher d(n,0.5) value compared with the freshly produced initial suspension. The amount of small spheres decreases in the suspension during the loading process. The filtrate shows a lower median d(n,0.5) value of 256 nm due to the filter effect of the e-PTFE layer which restrains spheres over approximately 300 nm in diameter.

### Anchoring

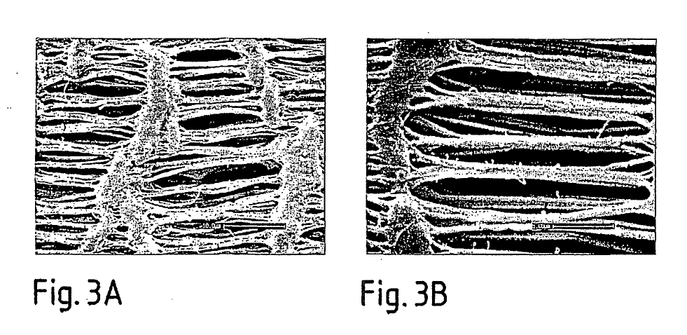

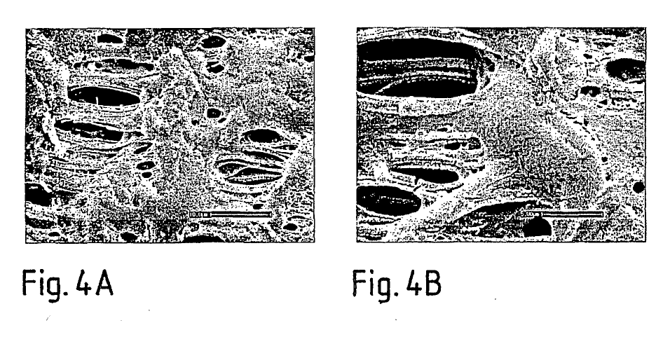

For anchoring the dexamethasone carried by the poly(D,L-lactide-co-TMC) (90:10)-nanospheres in an e-PTFE-matrix, the nanosphere-loaded e-PTFE foil was subsequently treated with supercritical CO₂ for 30 minutes under a pressure of 305 bar. REM pictures were taken, and are presented herein at two different magnification levels of 2500 fold (FIGS. 3A and 4A) and 5000 fold (FIGS. 3B and 4B), respectively, in FIGS. 3A, 3B without nanosphere-loaded e-PTFE and 4A, 4B with nanosphere-loaded e-PTFE. The nanospheres lose their spherical shape due to their solubility in supercritical CO₂.

The remaining e-PTFE matrix is insoluble in supercritical CO₂ and is surrounded by the dissolved poly(D,L-lactide-co-TMC).

Additionally, a release experiment of the hydrophobic model substance dexamethasone from poly(D,L-lactide-co-TMC) (90:10) was performed. Here, the ratio (90:10) refers to the weight ratio of lactide to TMC. Dexamethasone-loaded nanospheres were used for the release experiments. The median d(n=0.5) of the nanospheres was 216 nm.

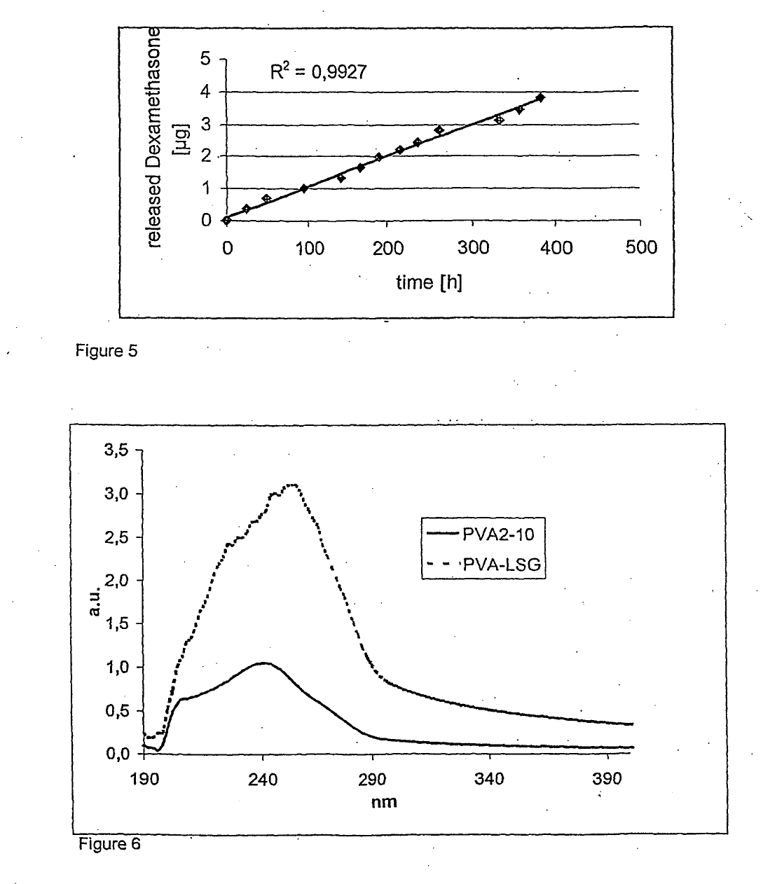

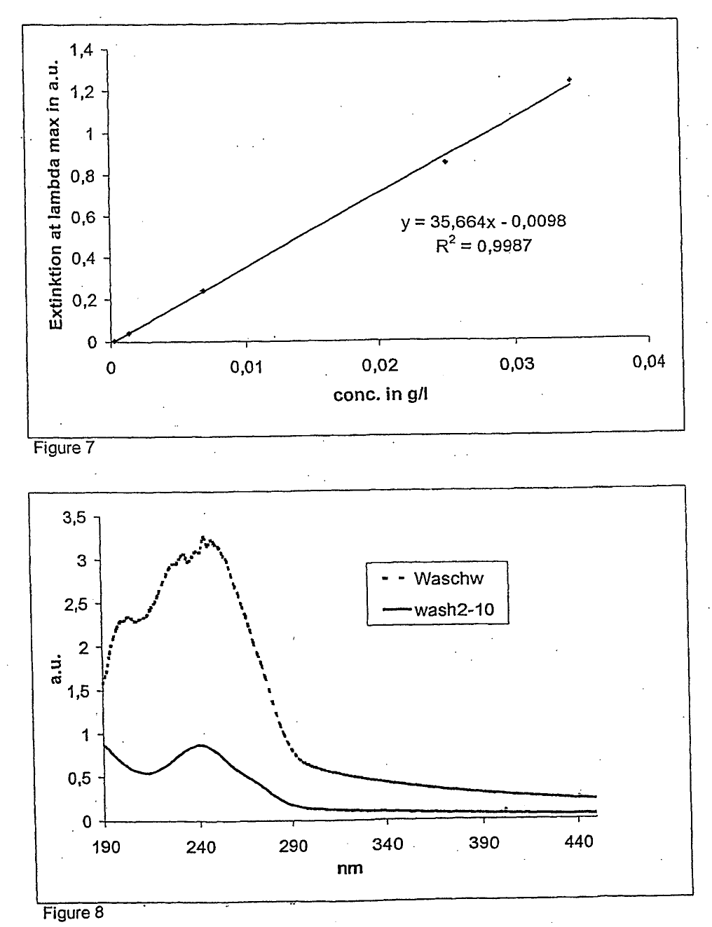

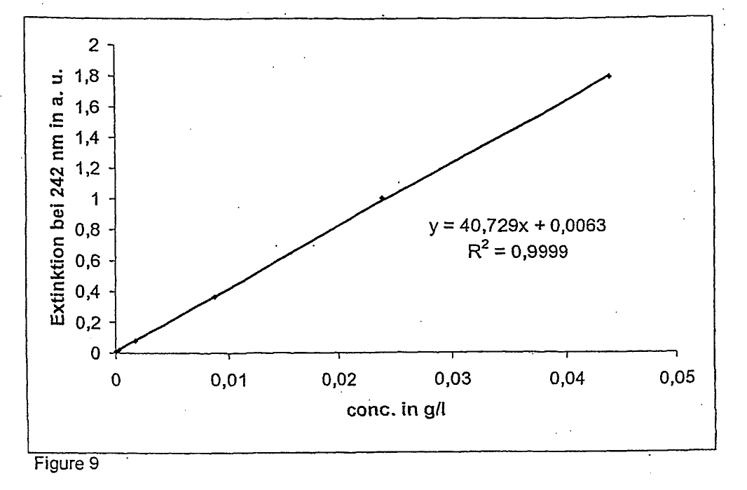

The nanospheres were made by the o/w solvent evaporation technique with a content of 20% (w/v) dexamethasone with respect to the initial amount of polymer. FIG. 5 shows the release pattern. The straight solid line indicates a line of best fit, the result of a least square analysis of the experimental data points. The variance of R²=0.9927 is also shown. The variance being very close to 1.0 and the very good agreement of the experimental data points with the straight line suggest that the rate at which dexamethasone is released from the nanospheres does not change with time. Therefore, a constant amount of dexamethasone migrates through the encapsulation of the nanospheres.

Next, the amount of dexamethasone incorporated into the nanospheres was determined, so that the effective drug loading could be calculated. The loading was determined to be 11.39%.

During the incorporation of drugs into nanoparticles according to the emulsion solvent evaporation process, a large quantity of the drug migrates into the aqueous PVA-solution. Also during the subsequent solvent washing, migration into the aqueous phase occurs. The content of dexamethasone in the PVA-solution and in the wash-water was determined using UV-spectroscopy. FIG. 6 depicts the UV-spectrum of the PVA-solution (solid line) used for the production of the nanospheres containing dexamethasone.

A 4% (w/v) PVA-solution (M=61.000) as a standard was measured. The measurement of the pure PVA-solution yielded pronounced extinctions. For this reason, the solution was diluted in a ratio of 2 to 8 (2 parts of dexamethasone containing PVA-solution to 8 parts of a 4% PVA-solution). Reference is made to FIG. 6 (curve PVA-2-10). The λₘₐₓ value of dexamethasone was determined to be 242.3 nm.

FIG. 7 shows the calibration line for dexamethasone in a 4% PVA-solution (M=61.000). The respective extinctions at λₘₐₓ were measured for different dexamethasone concentrations. The straight line indicates a least square analysis line of best fit of the experimental data points. The equation of the linear regression is also shown. Good agreement of the experimental values was obtained with the linear regression fit.

Subsequently, the content of dexamethasone in the wash water solution was measured.

FIG. 8 shows the UV-spectra of dexamethasone in the wash-water solution. The samples were measured with respect to water as a standard. The content of dexamethasone in the solution again yielded pronounced extinctions. The sample was diluted in a ratio of 2 to 8 (2 parts of wash water to 8 parts of distilled water).

FIG. 9 shows the calibration line for dexamethasone in distilled water. The respective extinctions at λₘₐₓ=242 nm were measured for different dexamethasone concentrations. The straight line indicates a least square fit of the experimental data points. The equation of the linear regression is also shown. Again, good agreement of the experimental values with the linear regression fit was obtained.

The concentration of dexamethasone in the PVA-2-10 sample was determined (c=29.53*10.sup.−3 g/l). Hence, the amount of dexamethasone in the diluted PVA-2-10 sample(=10 ml) amounts to 2.953*10⁻⁴ g. Consequently, the same amount exists in the 2 ml undiluted PVA-solution. 50 ml of the PVA solution were used during the production process and the amount of dexamethasone can be calculated (refer to Table 2). The amount of dexamethasone in the wash solution was calculated in the same manner.

TABLE 2
Calculation of the amount of dexamethasone in the
PVA-solution and in the wash-solution

| | Volume used | | | | |
|---|---|---|---|---|---|
| | in the production | Volume used for UV- | Dilution (UV- | | |
| | process | spectroscopy | spectroscopy) | Sample name | |
| PVA-solution | 50 ml | 2 ml | 2 ml + 8 ml | PVA-2-10 | |
| Wash- | 50 ml | 2 ml | 2 ml + 8 ml | wash-2-10 | |
| | | Formula from | | Mass of | |
| | Extinction | linear regression | | dexamethasone | |
| | at λ max | (FIG. 7 + 9) | Concentration | in 50 ml | |
| PVA-solution | 1.043 a.u. | X = (y − 0.0098)/35.664 | 29.53 * 10⁻³ g/l | 7.38 | mg |
| wash solution | 0.867 a.u. | X = (y − 0.0063)/40.729 | 21.13 * 10⁻³ g/l | 5.28 | mg |
| | | | | Σ12.66 | mg |

During production, 200 mg of the polymer and 40 mg of dexamethasone were used. 12.66 mg migrated into the aqueous phases, so it can be assumed that 27.34 mg of dexamethasone was incorporated into the spheres corresponding to 11.39%.

In the foregoing, the following abbreviations have been used:
o/w: oil in water
w/v: weight per volume
REM: Raster Electron Microscopy

The best mode of the invention has been described above only for the model substance dexamethasone. Examples of biologically-active substances to be anchored in an e-PTFE matrix instead of hydrophobic dexamethasone are: hydrophilic substances, such as acryflavine-hydrochloride; rapamycin (sirolimus); taxol (paclitaxel); NO donors or other agents directly or indirectly causing an increase of local NO concentration; growth factors to enhance a endothelialisation (e.g. VEGF); growth inhibitors; growth receptor antagonists; antithrombotic agents (e.g. heparin, GPIIb/IIIa receptor antagonists, hirudin); matrix metalloproteinase inhibitors; anti-inflammatory agents; antiproliferative agents (e.g. anti-tumor drugs, e.g. mitomycin C, 5-FU, cisplatin, gemcitabine, radioactive nuclides, adriamycine, mitoxantrone); antisense agents (ogligonucleotides or related compounds blocking transcription/translation process); antimigratory drugs; antioxidants; agents promoting or inhibiting apoptosis; genes for genetransfer (naked or packaged in vectors); hormones, somatostatin analogs; antibodies; angiogenesis inhibitors/promoters.

A supercritical CO₂ has been used for anchoring the biological active substance in the ePTFE foil. However, it is conceivable to use other supercritical agents, such as supercritical nitrogen, which do not harm the biologically active substance.

What is claimed is:

1. A method of impregnating an e-PTFE foil, comprising:
providing nanoparticles including a biologically-compatible substance and a surface layer that encapsulates the biologically-compatible substance;
suspending the nanoparticles in a fluid medium; and
imposing a pressure differential across an e-PTFE foil to urge the fluid medium into interstices of the e-PTFE foil.

2. The method according to claim 1, wherein the pressure differential is in a range up to approximately 10 bar.

3. The method according to claim 1, further comprising the step of anchoring the nanoparticles in the e-PTFE foil interstices.

4. The method according to claim 3, wherein the anchoring step comprises. agglomerating the nanoparticles.

5. The method according to claim 4, wherein the agglomerating step comprises the step of heating the foil.

6. The method according to claim 3, wherein the anchoring step comprises contacting the nanoparticles with a liquid agent selected to dissolve the surface layer.

7. The method according to claim 6, wherein the liquid agent comprises supercritical CO₂.

8. The method according to claim 7, further comprising the step of treating the e-PTFE foil with supercritical CO₂.

9. The method according to claim 8, wherein the step of treating the e-PTFE foil includes treating for approximately 30 minutes at a pressure of approximately 305 bar.

10. The method according to claim 1, wherein the providing step comprises preparing the nanoparticles by an oil in water emulsion solvent evaporation technique.

11. The method according to claim 10, wherein the evaporation technique includes mixing a polymer with the biologically-compatible substance to form a first mixture, cooling the first mixture, adding an aqueous solution to the first mixture to form a second mixture, dispersing the second mixture to form an emulsion, and removing the organic phase of the emulsion.

12. The method according to claim 11, wherein the biologically-compatible substance comprises dexamethasone, and wherein 200 mg of the polymer is mixed with 40 mg of dexamethasone in 5 mL of methylenecholoride to form the first mixture.

13. The method according to claim 12, wherein the aqueous solution added to the first mixture to form the second mixture comprises 4% (w/v) polyvinylalcohol, and wherein the second mixture is dispersed by vortex.

14. The method according to claim 13, further comprising the step of anchoring the nanoparticles in the e-PTFE foil interstices.

15. The method according to claim 14, wherein the anchoring step includes the step of treating the e-PTFE foil with supercritical CO₂.

16. The method according to claim 15, wherein the step of treating the e-PTFE foil includes treating for approximately 30 minutes at a pressure of approximately 305 bar.

17. The method according to claim 1, wherein the nanoparticles comprise a polymer selected from the group consisting of poly(D,L-lactide), poly(D,L-lactide-co-glycolide), poly(D,L-lactide-co-trimethylenecarbonate, and combinations thereof, the providing step comprising dissolving the polymer in a solvent.

18. The method according to claim 17, wherein the dissolving step comprises providing a water-miscible solvent and utilizing a solvent displacement method.

19. The method according to claim 17, wherein the dissolving step comprises providing a water-non-miscible solvent and utilizing a solvent evaporation method.

20. A method of impregnating an e-PTFE foil, comprising:
providing nanoparticles including a biologically-compatible substance and a biodegradable surface layer that encapsulates the biologically-compatible substance;
suspending the nanoparticles in a fluid medium;
imposing a pressure differential across an e-PTFE foil to urge the fluid medium into interstices of the e-PTFE foil; and
anchoring the nanoparticles in the e-PTFE foil interstices.
